(19) United States
(12) Patent Application Publication
(10) Pub. No.: US 2007/0141655 A1
(43) Pub. Date: Jun. 21, 2007

(54) Multi symptom medical test to diagnose causes of vaginitis

(75) Inventors: Kuo Chiang Yee, Mercer Island, WA (US); Hsian Chiang Yee, Mill Creek, WA (US); Hsian Pei Yee, Mill Creek, WA (US)

Correspondence Address: EMIL CHANG;LAW OFFICES OF EMIL CHANG, 874 JASMINE DRIVE, SUNNYDALE, CA, 94086, US

(73) Assignee: AmeriTek Research LLC

(21) Appl. No.: 11/303,268

(22) Filed: Dec. 16, 2005

(51) Int. Cl. G01N 33/00

(52) U.S. Cl. 435/7.93

(57) ABSTRACT

The present invention provides methods and apparatuses for a multi symptom test system. The multi symptom test apparatus having a plurality of zones for testing a plurality of conditions comprises the plurality of zones configured to receive a test sample wherein the zones responsive to the test sample provides a result indicative of a particular vaginitis test result.

### FIELD

The present invention relates to multi symptom testers and, more particularly, to method and apparatuses for a multi result zone tester that focuses on a combination of test properties to provide accurate diagnosis of conditions.

### BACKGROUND

Vaginitis refers to any inflammation or infection of the vagina. Women of all ages find this to be a common gynecological problem with most women having at least one form of vaginitis at some time during their lives. In the United States, vaginal infections represent the most common reason women visit their gynecologists, accounting for more than 10 million office visits each year.

When the walls of the vagina in a woman become inflamed because some irritant has disturbed the balance of the vaginal area, vaginitis occurs. Bacteria, yeast, viruses, and/or chemicals in creams or sprays, or even clothing contribute to manifestations of vaginitis. Vaginitis often occurs from organisms that are passed between sexual partners. Additional factors that affect the vaginal environment include the overall health of the woman, personal hygiene, medications, hormones (particularly estrogen), and the health of her sexual partner. A disturbance in any factor or combination of these factors can trigger vaginitis.

Common symptoms of vaginitis are pain, itching or burning inside the vagina and also on the skin or vulva. Abnormal vaginal discharge and discomfort during urination or sexual intercourse may also occur. If everyone with vaginitis had these symptoms, then the diagnosis would be fairly simple. However, as many as four out of every 10 women with clinically diagnosable vaginitis may not have these typical symptoms. In addition, any symptoms are usually aggravated by sexual intercourse. A discussion of the various specific disease states follows.

Bacterial vaginitis (BV), the most common vaginitis, has been associated with pelvic inflammatory disease (PID), cervicitis, postoperative infection, abnormal cytology (cellular structure), and increased acquisition of human immunodeficiency virus (HIV) infection and other sexually transmitted diseases (STDs). BV can also cause obstetric complications such as preterm labor and low-birth-weight infants.

Common symptoms for BV include: a milky, thin discharge at times or a heavy, gray discharge and a “fishy” odor which may become more noticeable during inter-course. However, any individual may experience symptoms differently.

Yeast infections, as they are commonly called, are caused by a species of fungus known as Candida, which normally lives in the vagina in small numbers. Candida can also be present in the mouth and digestive tract in both men and women.

Since yeast is normally present and well balanced in the vagina, infection occurs when something in the system of the women upsets this normal balance. For example, an antibiotic for bacterial infection may upset the yeast balance of the vagina. In this case, the antibiotic kills the bacteria that normally protects and balances the yeast in the vagina. In turn, the yeast overgrows, causing a vaginal infection. Other factors that can cause this imbalance to occur include pregnancy, which changes hormone levels, and diabetes, which allows too much sugar in the urine and vagina.

Yeast infections cause a thick, white vaginal discharge with the consistency of cottage cheese. Although the discharge can be somewhat watery, it is often odorless. Yeast infections usually cause the vagina and the vulva to be very itchy and red.

Trichomoniasis is a sexually transmitted infection. Trichomonal vaginitis is caused by a one-celled parasite called Trichomonas vaginalis, which passes between partners during sexual intercourse. It can also survive on objects with moisture and is therefore not constrained to sexual transmission. Because most men do not present symptoms with trichomoniasis, the infection is often not diagnosed until the woman develops symptoms of vaginitis. When this organism infects the vagina, it can cause a frothy, greenish-yellow discharge. Women with Trichomonal vaginitis may complain of itching and soreness of the vagina and vulva, as well as burning during urination. In addition, there can be discomfort in the lower abdomen and vaginal pain during intercourse; however there may be no other symptomology.

Trichomonal vaginitis is widespread throughout the world. This form of vaginitis presents in 50 percent of women, approximately three million cases per year within the United States alone.

Viruses are a common cause of vaginitis, with most being spread through sexual contact. One type of virus that causes viral vaginitis is the herpes simplex virus (HSV, or simply herpes), whose primary symptom is pain in the genital area associated with lesions and sores. These sores are generally visible on the vulva, or vagina, but occasionally are inside the vagina and can only be found during a pelvic examination. Stress or emotional situations are often found to be a factor in triggering an outbreak of herpes.

Another source of viral vaginitis is the human papillomavirus (HPV), a virus that is also transmitted through sexual contact. This virus causes painful warts to grow on the vagina, rectum, vulva, or groin. However, visible warts are not always present, in which case, the virus is generally detected by a Pap test.

Another primarily sexually transmitted form of-vaginitis is caused by the bacterium Chlamydia trachomatis. Unfortunately, many women experience no symptoms. This makes diagnosis difficult, thus prolonging detection and treatment, as well as raising the probability of further spreading of the disease. The following are the most common symptoms of Chlamydia: increased vaginal discharge; light bleeding, especially after intercourse; pain in the lower abdomen or pelvis; burning during urination; pus in the urine; and redness and swelling of the urethra and labia. However, each individual may experience symptoms differently. Since the symptoms of Chlamydia may resemble other vaginitis conditions, misdiagnosis can occur.

Misdiagnosed, incorrectly diagnosed, or undiagnosed vaginitis creates a host of consequent medical issues. For example, a recent study of 771 women who had conceived through in vitro fertilization revealed that of the 190 women diagnosed with Bacterial Vaginitis (BV), 31.6% sustained a miscarriage during the first trimester, compared with 18.5% of those women without BV. Furthermore, this increased risk remained significant after adjusting for other risk factors. Although the exact cause of an association between BV and miscarriage is unclear, researchers hypothesized that endometritis may be a factor due to the early timing of the miscarriages. The majority of miscarriages in the study occurred within the first six weeks of conception, suggesting a failure of implantation or early embryonic development, both of which have been linked to endometritis. Previous studies have found an association between BV and an increased risk of endometritis, along with several other gynecologic and obstetrical complications including postoperative infection, cervicitis, cervical intraepithelial neoplasia (CIN), HIV, premature rupture of membranes, and preterm labor and delivery.

Additional studies show that one quarter to one half of all Americans may acquire human papillomavirus (HPV), a STD that has been linked to cervical cancer and is the cause of genital warts. Appearing in the genital and perianal areas in both men and women, most warts are asymptomatic, but some cause itching, burning, pain and tenderness. They also may cause emotional stress, especially if they appear in one partner of a monogamous relationship. Though warts usually incubate from six weeks to three months, often the virus lies dormant for an indeterminate period of many years.

It is clear that misdiagnosed, incorrectly diagnosed, or undiagnosed vaginitis is a major health issue that calls for the development of successful diagnosis and treatment modalities.

Current methods of diagnosing vaginitis are few: routine examination, including pH tests and KOH evaluation, and microscopic evaluation. Recently, a few commercial test kits have been introduced into the marketplace.

A survey indicated that most physicians, when presented with potential vaginitis cases, only routinely perform microscopy, while pH and KOH tests were performed only very occasionally (citations at end of this monograph). The survey also found that lack of proper diagnosis might lead to inappropriate treatment of vaginitis; for instance, in many cases, treatment was prescribed when there was no objective evidence of disease.

Clinicians should be sensitive to the fact that women may not be communicating symptoms because they may believe that they are asymptomatic. For instance, the presence of vaginal odor is considered by many women to be normal or a minor hygiene problem at worst. Therefore, vaginal odor may not be communicated to the health care provider as a possible symptom, and the physician may not pursue it.

Unfortunately, most patient's understanding of vaginitis is usually very low. Surveys have found that, while most American women have heard of yeast infection, relatively few are familiar with BV or trichomoniasis. Moreover, published studies have consistently demonstrated that most women fail to accurately self-diagnose vaginal infections, and many may wrongly use over-the-counter yeast treatments, which are ineffective in treating more common forms of vaginitis.

There is a close link between vaginal health and pH. Fluctuations in vaginal pH are often normal occurrences resulting from variations during the menstrual cycle, a decrease in pH during pregnancy, or the presence of seminal fluid, blood, amniotic fluid, or cervical mucus. However, an abnormal pH level may also indicate the presence of a vaginal infection, such as BV.

In a healthy vagina, lactobacilli-the main determinant of pH-are instrumental in maintaining vaginal acidity (normal pH is 3.8 to 4.2), and preventing the overgrowth of other aerobic and anaerobic bacteria. In BV, the pH is elevated in association with an increase in microorganisms that impair the growth of lactobacilli. Because of the critical role pH plays in suppressing the growth of BV-associated bacteria, an increased vaginal pH is an important indicator in diagnosing BV.

Most physicians accept the criteria of pH>4.5 or 4.7 as one indicator of BV. Regardless of the level used, if a higher than normal vaginal pH is detected, other diagnostic tests should be performed to investigate the presence of BV or other infections. In order to ensure the accuracy of the pH test, obtaining a reliable vaginal fluid sample is essential. A specimen should be collected from the anterior fomix or lateral vaginal wall. It is important that contact with the cervix is avoided because cervical pH is higher than vaginal pH. The accuracy of pH tests may also depend on the method or equipment used. In studies, bench pH meters and handheld pH meters have high accuracy rates; however, in the clinical setting, these tests may be impractical. Although pH paper may be less accurate, it is a practical approach for detecting increases in vaginal pH above 4.5 and higher. Additionally, pH paper is inexpensive and convenient because it can be placed directly on the vaginal wall. Initial screening for vaginitis in the office or clinic can therefore be accomplished by a three-step testing procedure on a single sample of vaginal discharge. The testing requires pH paper, a dilute solution of potassium hydroxide (KOH), saline solution and a microscope for a wet mount, and may be performed routinely by appropriate support staff in the office. This testing procedure can provide health benefits from an initial diagnosis, as well as long-term savings by ensuring proper treatment and prevention of more serious conditions associated with BV, trichomoniasis, or candidiasis.

The first step is to use pH paper to measure the acidity of the discharge sample. While the normally acidic pH of the vagina is approximately 4.0, a more alkaline pH level greater than 4.5 may indicate BV or trichomoniasis.

The second step involves adding KOH to the discharge sample to intensify and more easily detect any odor. A foul or fishy odor is associated with BV and sometimes trichomoniasis.

The third step involves preparing a wet mount by adding saline solution to a woman's vaginal secretions and viewing the slide under a microscope. BV is indicated by the presence of clue cells—pithelial cells coated with bacteria. Yeast infection is apparent on viewing an overgrowth of yeast organisms and trichomoniasis is indicated when trichomonad parasites are present. If a vaginal discharge sample has an alkaline pH reading of more than 4.5, and/or if a fishy amine odor is released on combining a sample with KOH, BV or trichomoniasis may be present. However, if these tests are negative—the pH reading is ≦4.5 and the KOH test releases no amine odor—but a discharge and/or itching or burning is present, there may be a yeast infection.

In either case, microscopic examination can then be undertaken to pinpoint the specific infection. The wet mount is prepared by adding saline solution to a sample of the patient's discharge and viewing the sample under a microscope. A normal vaginal discharge sample will show lactobacilli and epithelial cells. Clue cells with adherent coccoid bacteria but no white blood cells are the marker for BV. Trichomonads and more than 10 white blood cells per high-power field will appear under the microscope if trichomoniasis is present. When KOH is added to the discharge sample, budding yeast, hyphae and pseudohyphae indicate candidiasis.

Viewing wet-mount slides is a fairly reliable method of diagnosing BV if used properly by trained personnel. Although use of the wet-mount examination for clue cells is an important clinical test for diagnosing BV, wet-mount evaluations can be technically difficult, and the results can be dependent on the observational skills of the clinician. Therefore, wet-mount results have been used in conjunction with other test results to confirm a vaginal infection.

Several points emerge from this short review of the vaginitis diagnostic standards of care. First, diagnostic methods in use today are cumbersome, invasive and time consuming. In a busy office or clinic, it is easy to miss some of the procedures, leading to uncertain results. Finally, the diagnostic results are obtained by generally subjective evaluations, and are thus hostage to the level of competence of the practitioner in the individual clinic or professional office.

Vaginal infection can be identified by a three-step in-office procedure using a single sample of vaginal discharge. The use of pH paper, potassium hydroxide solution (KOH), saline solution and a wet mount microscope may be performed immediately by appropriate support staff in the office with no culture. In the hands of a trained and experienced technician, this test procedure may be quite effective. However, it is time consuming and subjective, and thus errors may be made.

A few commercially available products may assist the office or clinic in the diagnosis of vaginitis by improving on the diagnostic weaknesses of the three step process and removing the dependence on the subjective evaluation skills of lab personnel.

FemExam® is a diagnostic card including both pH and amine indicators. About the size of a credit card, FemExam® is simple to use, requiring the clinician to use a swab to apply a sample of vaginal discharge to the test area on the card. Each test uses an easy-to-read plus sign to indicate a positive outcome. Results appear almost immediately. Self-obtained vaginal swabs have been shown to be reliable specimens for use with this product.

Another product accomplishes part of this task. Care Plan® pH gloves are designed to measure vaginal pH. Self-measurement of vaginal pH is effective for assisting in the identification of BV. This is significant because women with BV may be at risk for delivering prematurely and premature rupture of membranes.

Commercially available test paper is also a quick and inexpensive method of determining vaginal pH levels. A pH meter can also be used, although this tool is more costly than pH paper. Regardless of the method used to determine pH levels, if an elevated pH is detected, a diagnosis must still be confirmed. The presence of blood, seminal fluid, and amniotic fluid can all affect the pH levels in the vagina.

Another product, Affirm III® is a DNA probe test that detects Candida species (yeast), Gardnerella vaginitis (the bacteria most frequently associated with BV), and Trichomonas vaginitis in a single vaginal sample. The test takes about 45 minutes to produce a result. Becton-Dickinson, the manufacturer of the Affirm III probe, boasts an accuracy rate that is 30% higher than that obtained from the use of wet-mount slides, especially when there are mixed infections. The test equipment is very costly, and so the test is expensive to perform on all patients.

It is obvious from this short list of products designed to evaluate vaginitis, and the cumbersome and error prone office procedures commonly used, that a comprehensive multi symptom test kit that easily and accurately diagnoses vaginitis would be very welcome on the market.

Other aspects and advantages of the present invention will become apparent to those skilled in the art from reading the following detailed description when taken in conjunction with the accompanying drawings.

### BRIEF DESCRIPTION OF THE DRAWINGS

FIG. 1 illustrates a test kit in accordance to an embodiment of the present invention;

FIG. 2 illustrates an example of a patient about to insert a sterile swab into her vagina;

FIG. 3 illustrates a first swab saturated with fluid sample to be applied to sample application zone 18 in accordance with an embodiment of the present invention;

FIG. 4 illustrates a second swab saturated with fluid sample to be applied to sample application zones 20 and 22 in accordance with an embodiment of the present invention;

FIG. 5 illustrates capping of the buffer solution container after a third swab saturated with fluid sample has been mixed with the buffer solution;

FIG. 6 illustrates the buffer solution container being shaken vigorously up and down; and

FIG. 7 illustrates the mixed buffer solution being applied to sample application zones.

### DETAILED DESCRIPTION

As disclosed below, the present invention provides methods and apparatuses for a multi symptom test kit to replace the expensive and cumbersome microscopic evaluations and laboratory culture procedures. In general, the preferred embodiment of the present invention is a multi test zone test kit, with a total of six specific biochemical evaluations on one test platform. The test kit has a total of seven sample application zones on the small test platform. Each specific biochemical evaluation test zone produces color changes for each positive and negative result. There are seven zones since one evaluation requires two zones. There are six individual chemical and biological tests that screen for specific chemical or biological aspects of the vaginal fluid samples. The six result zones can accurately differentiate between the various disease states that are normally found in vaginitis.

According to an embodiment of the present invention, vaginal fluid samples are collected in a three-step collection procedure; two samples are used directly on test zones, and the third is diluted in a custom designed buffer dilution and delivery system. The test results are determined by observing color changes on each test zone following sample application. The colors are compared to a color chart on the test cassette to draw conclusions as to the results of each test. A diagnostic protocol to accurately determine the condition causing the vaginitis is then consulted to make the final determination.

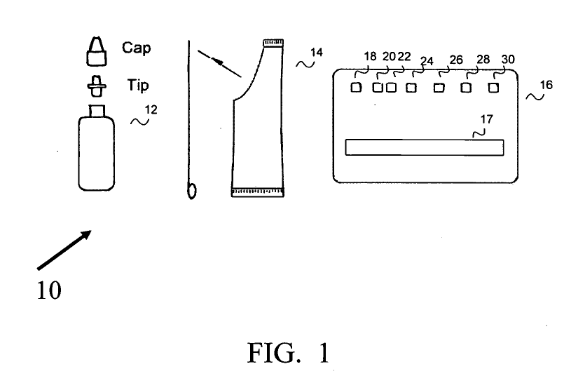

FIG. 1 illustrates an embodiment of a multi symptom test kit 10. The test kit 10 includes a buffer solution container 12 having a tip and cap, sterile swabs 14, and a test tray 16. The test tray 16 includes a color chart 17 and seven sample application zones 18, 20, 22, 24, 26, 28, and 30. The sample application zone 20 is used in conjunction with sample application zone 22 to provide a test result in the sample application zone 22. Sample application zones 18, 22, 24, 26, 28, and 30 provide results in response to a sample applied. Accordingly, the sample application zones 18, 22, 24, 26, 28, and 30 are also referred to result zones 18, 22, 24, 26, 28, and 30.

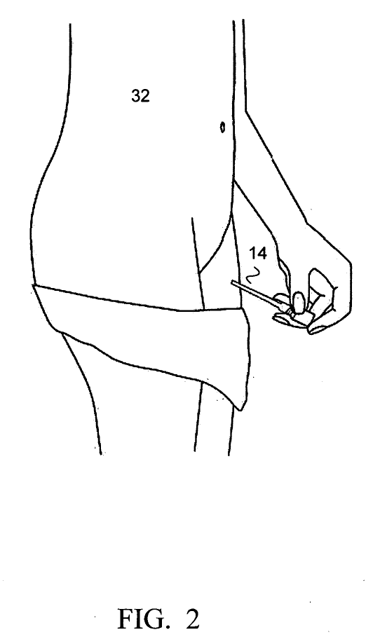

FIG. 2 illustrates an example patient 32 about to insert a sterile swab 14 into her vagina. The patient or medical personnel should gently open the vaginal opening and insert the swabs about two to three inches into the vagina. It is preferable that the sterile swab 14 not be inserted near the cervix as the pH results may be inaccurate. It is understood that the patient or the medical personnel posses the skill and knowledge to differentiate the cervix and avoid insertion of the sterile swab 14 close to the cervix. Once the sterile swab 14 is inserted in the vagina, gently stroke the inner walls of the vagina with the each of the three swabs 14, ensuring that the swabs are all moistened thoroughly. Leave the swabs in the vagina several minutes to ensure they are saturated with vaginal fluid.

Next, take the buffer solution container 12 and the test tray 16. It may be desirable to write the patient's name in a space provided on the test tray 16 for future reference.

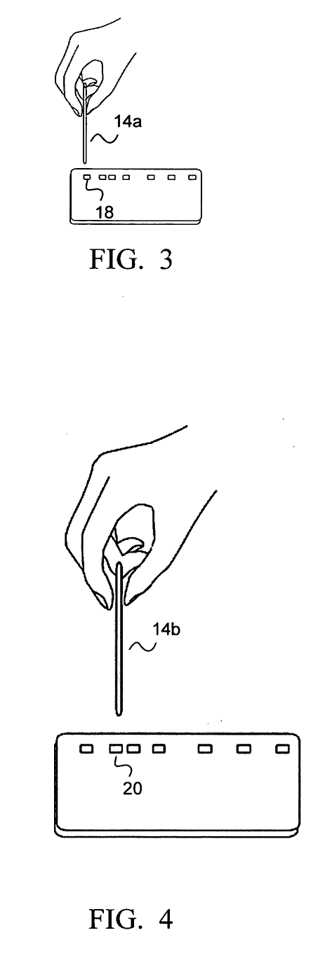

After removing the three swabs 14 from the vagina and referring to FIG. 3, rub a first swab 14a saturated with fluid sample onto sample application zone 18—the pH zone—of the test tray 16. Discard the first swab 14a in a biological specimen container (not shown). Read the pH color after three minutes from the application or result zone 18, and note the result on the test tray 16 next to the pH zone result color comparison of color chart 17. It may be desirable to circle the color on the tray 16—either positive or negative—with an ink pen.

In accordance to an embodiment of the present invention, while the pH Zone color is developing take the second swab 14b and rub onto sample application zone 20 six or seven times. Then, immediately rub this same swab onto sample application zone 22 several times. Wait 1-5 minutes. If the second swab 14b or sample application zone 22 becomes peach or pink within one minute, it is a positive reading for Gardnerella. It may be desirable to record the Gardnerella result on the test tray 16 near the Gardnerella Zone Result color comparison of the color chart 17.

Unscrew cap 34 to open the buffer solution container 12, with the third swab 14c thoroughly mix the swab 14c into the buffer solution container.

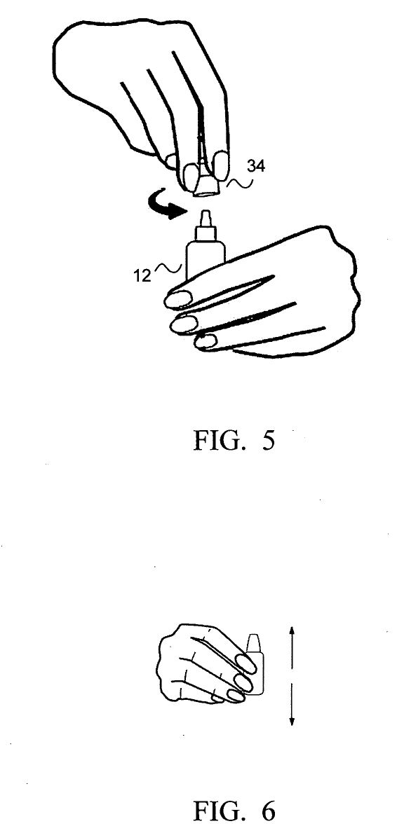

It is desirable to swirl the swab vigorously for approximately 15 seconds, and then expunge as much liquid as possible from the swab by pressing and rotating the fiber portion against the wall of the solution container 12. Discard the swab. Screw dropper tip 36 securely onto the buffer solution container 12 with vagina fluid specimen. Then screw the cap 34 onto the buffer solution container 12 with vagina fluid specimen. (FIG. 5).

Referring to FIG. 6, after the specimen container 12 with vagina fluid specimen is tightened securely, it is desirable to shake vigorously up and down ten (10) times.

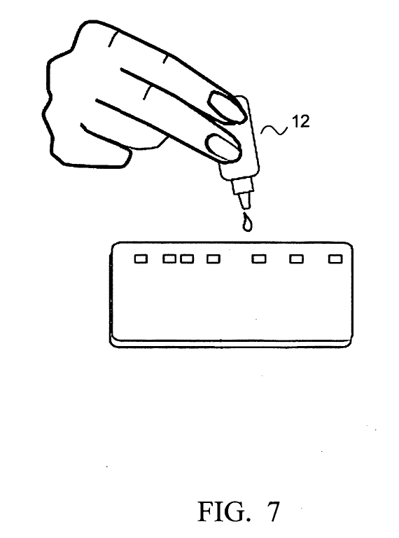

Referring to FIG. 7, squeeze the buffer solution container 12 with vagina fluid specimen above each of the sample application zones 22, 24, 26, 28, and 30 preferably using one drop on each of the sample application zones 22, 24, 26, 28, and 30.

Within about two minutes, color results appear in each of the sample application or result zones 22, 24, 26, 28, and 30.

It may be desirable to promptly circle the square that most closely matches each result zone color with a corresponding color on the color chart 17. In accordance with an embodiment of the present invention, it is also desirable to compare the result zone colors with the corresponding color on the color chart 17 within two (2) to three (3) minutes after placing the droplets of the buffer solution container 12 with vagina fluid specimen onto the sample application zones 22, 24, 26, 28, and 30.

Finally, it is preferable that the buffer solution container 12 with vagina fluid specimen, the second and third swabs 14b, 14c be placed in a biological specimen container (not shown).

In accordance with the present invention, it is desirable that the specimen used for pH testing must be an undiluted vaginal fluid sample; the specimen used for Gardnerella testing must be a separate undiluted sample; the specimen used for the remaining tests must be diluted in the buffer solution container as directed; the collection of the vaginal fluid sample should be performed in a single step; ensure that nothing remains inside the vagina after collection; and read the colors on the test tray within the prescribed time of application of the sample, and circle the color result with a pen on the test tray to record the results.

The characteristic and an example composition of each of the sample application zones is discussed below to enable those skilled in the art build the multi symptom test apparatus.

Since the normal pH of vaginal fluid is in the range 3.8-4.2, after application of the test fluid sample, if the pH test zone (sample application zone 18) turns from pink to light blue-green within 3 minutes, the pH is above 4.7, which indicates a positive result. If the vaginal fluid is below pH 4.7, the color remains pink, indicating normal vaginal pH. The pH zone change to a light blue-green color—an abnormally high pH—is a positive finding, consistent with bacterial vaginitis and/or trichomoniasis, microorganisms that impair the growth of the normal vaginal lactobacilli, which keep pH low.

An example of an indicator that is useful for the positive control when the pH indicator lamina has a transition point of 4.7 is a mixture of bromothmol blue and bromocresol green. The weight ratio of bromothmol blue and bromocresol green in such a mixture can vary and different ratios will produce different transition points.

An enzyme activity test specifically designed to detect the presence of Gardnerella vaginalis bacteria and a few other infectious bacteria in vaginal fluid specimens is used in Zone 22. The development of a visible peach-to-pink-to-red color on the test swab after application of the vaginal fluid sample onto the result zone 22 is a positive test result, indicating the presence of Gardnerella Bacterial vaginitis. No color change on the test swab indicates there is no Gardnerella infection.

It is discovered that enzymatically active proline iminopeptidase (alternatively, proline aminopeptidase), other enzymes exhibiting proline iminopeptidase activity, and enzymatically active hydrolases in general, which are present in unprocessed or minimally processed vaginal fluid or any other liquid sample, can be detected in a rapid, simple and accurate manner. For proline iminopeptidase activity, the assay is useful for point-of-care detection and diagnosis of bacterial vaginosis.

Sample application zone 24 depends upon the chemical conversion of nitrate to nitrite by the action of gram-negative bacteria in the vaginal fluid. If the test procedure using the buffer diluted vaginal fluid sample turns the test zone from colorless to a pink color, this is a positive reaction consistent with the presence of a yeast infection. If no color change is observed, this indicates a lack of yeast.

Most of the bacteria which are present in the case of infections reduce the nitrate in the bladder into nitrite. A known acid sensitive method of the detection of bacteria depends upon the determination of the amount of nitrite in an acid solution. The nitrite diazotized the asanilica acid and the diazooim salt couples with another ingredient to give a red azo dyestuff. The composition of the sample application zone 24 is approximately: 2.0% w/w p-arsanilic acid, 2.2% w/w a-naphthylamine and 95.8% w/w buffer. A vaginal infection may result in bleeding in the vaginal cavity. After application of the buffer diluted vaginal fluid sample onto the blood zone or result zone 26, a color change from yellow to dark green or blue is an indication of blood in the vaginal fluid. The presence of blood indicates the possibility of a Chlamydia infection or severe Bacterial vaginitis. A confounding factor can be the presence of menstrual blood in the sample, which may result in a false positive test. If menstrual blood may be present, this zone should be given less weight in the diagnostic scheme.

A number of test systems for the determination of occult blood are known. These test systems rely on the detection of the peroxidase activity of hemoglobin. Such test systems are known in the form of test strips. Such strips typically include an indicator or chromogen an a hydroperoxide compound in addition to various buffers and stabilizers. In accordance with an embodiment of the present invention, the sample application zone 26 has the following composition: 6.6% w/w cumen hydroperoxide, 2.0% w/w 3,3′,5,5′ tetramethylbenzidine, and 91.4% w/w non-reactive ingredients.

Application of the buffer diluted vaginal fluid sample onto the Protein Zone or sample application zone 28 results in a blue color if the protein concentration in the vaginal fluid exceeds normal levels. A blue color, a positive reaction, is consistent with the presence of Chlamydia or Bacterial vaginitis, but also may occur with other forms of vaginitis. The infectious organisms produce pus, which will result in abnormal protein levels in the vaginal fluid.

In order to determine if an individual has an albumin deficiency and/or to determine if an individual excretes an excess amount of protein, and in order to monitor the course of medical treatment to determine the effectiveness of the treatment, simple, accurate and inexpensive protein detection assays have been developed. Furthermore, of the several different assay methods developed for the detection and/or measurement of protein in urine and serum, the methods based on dye binding techniques have proven especially useful because dye binding methods are readily automated and provide reproducible and accurate results.

In general, dye binding techniques utilize pH indicator dyes that are capable of interacting with a protein, such as albumin, and that are capable of changing color upon interaction with a protein absent any change in pH. When a pH indicator dye interacts with, or binds to, a protein, the apparent pKₐ (acid dissociation constant) of the indicator dye is altered and the dye undergoes a color transition, producing the so-called “protein-error” phenomenon. In methods utilizing the dye binding technique, an appropriate buffer maintains the pH indicator dye at a constant pH to prevent a color transition of the pH indicator dye due to a substantial shift in pH. Due to the “protein-error” phenomena, upon interaction with the protein, the pH indicator dye undergoes a color transition that is identical to the color change arising because of a change in the pH. In accordance with an embodiment of the present invention, the sample application zone 28 has the following composition: 1.5% w/w tetrabromphenol blue and 98.5% w/w non-reactive ingredients.

Application of the buffer diluted vaginal fluid sample onto the leukocyte zone or sample application zone 30 results in a color change to pink or light purple if white blood cells are present. Light purple indicates a positive result, indicating a Trichomonas or Chlamydia infection, depending on the status of other test zones. If there is no color change, this indicates no Trichomonal or Chlamydia infections.

Methods for determining hydrolytic analytes included chromogenic esters which, when hydrolyzed by esterase or protease, produced a colored alcoholic product, the intact ester being of a different color from the free alcohol. Many of these systems included accelerator compounds and diazonium salt coupling agents.

Chemistries utilizing such esters are abetted by various hydrolysis accelerators, as well as diazonium salt coupling agents. In accordance with an embodiment of the present invention, the sample application zone 30 has the following composition: 0.1% w/w ester, 0.6% w/w diazonium salt, 40% w/w buffer and 59.3% w/w non-reactive ingredients.

A multi symptom test apparatus has been disclosed. Those skilled in the art will understand that the multi symptom test apparatus with many result zones tests for and diagnoses specific properties contained in the vaginal fluid of a woman. By focusing on these particular properties, an accurate diagnosis for vaginitis is obtained in a short time as opposed to visiting a clinic and waiting as long as 15 days to obtain results from cultures.

While the foregoing detailed description has described several embodiments of the present invention, it is to be understood that the above description is illustrative only and not limiting of the disclosed invention. Obviously, many modifications and variations will be apparent to those skilled in the art without departing from the spirit of the invention.

We claim:

1. A multi symptom test apparatus having a plurality of zones for testing a plurality of vaginitis conditions, comprising:
the plurality of zones configured to receive a test sample wherein the zones responsive to the test sample provides a result indicative of a particular vaginitis test result.

2. The multi symptom test apparatus according to claim 1, wherein the test sample includes a solution of buffered vaginal fluid.

3. The multi symptom test apparatus according to claim 1, wherein the test sample includes undiluted vaginal fluid.

4. The multi symptom test apparatus according to claim 3, wherein the test sample includes a solution of buffered vaginal fluid.

5. The multi symptom test apparatus according to claim 4, wherein:
a first plurality of zones is configured to receive a sample of the vaginal fluid; and
a second plurality of zones is configured to receive a sample of the buffered vaginal fluid.

6. The multi symptom test apparatus according to claim 5, wherein the first plurality of zones includes a pH zone configured to indicate a pH above or below 4.7 in the test sample.

7. The multi symptom test apparatus according to claim 5, wherein:
the first plurality of zones includes a first subzone and a second subzone; and
the sample vaginal fluid is contacted with the first subzone and the second subzone to indicate a result in the second subzone for presence or lack of presence of Gardnerella bacteria in the test sample.

8. The multi symptom test apparatus according to claim 5, wherein the second plurality of zones includes a nitrite test zone configured to indicate presence or lack of presence of nitrite in the test sample.

9. The multi symptom test apparatus according to claim 5, wherein the second plurality of zones includes a blood test zone to indicate presence or lack of presence of red blood cells in the test sample.

10. The multi symptom test apparatus according to claim 5, wherein the second plurality of zones includes a protein test zone to indicate presence or lack of presence of protein in excess of normal values in the test sample.

11. The multi symptom test apparatus according to claim 5, wherein the second plurality of zones includes a leukocytes test zone to indicate presence or lack of presence of white blood cells in the test sample.

12. A method for a multi symptom vaginitis test apparatus having a plurality of zones, comprising the steps:
contacting a test sample to the plurality of zones; and
observing a color change in the zones to provide an indication of a particular vaginitis test result.

13. The method for a multi symptom test apparatus according to claim 12, wherein the test sample include vaginal fluid and further comprises the step of collecting vaginal fluid.

14. The method for a multi symptom test apparatus according to claim 13, wherein the test sample include buffered vaginal fluid and further comprises the step of buffering the vaginal fluid.

15. The method for a multi symptom test apparatus according to claim 14, wherein the plurality of zones includes a pH zone and further comprises the step of contacting the vaginal fluid with the first zone to determine whether a pH of the vaginal fluid is within a predetermined range.

16. The method for a multi symptom test apparatus according to claim 14, wherein the plurality of zones includes a first subzone and a second subzone and further comprises the step of contacting the vaginal fluid with the first subzone and the second subzone to determine whether Gardnerella bacteria is present in the vaginal fluid.

17. The method for a multi symptom test apparatus according to claim 14, wherein the plurality of zones includes a nitrite zone and further comprises the step of contacting the buffered vaginal fluid with the nitrite zone to determine whether nitrite is present in the buffered vaginal fluid.

18. The method for a multi symptom test apparatus according to claim 14, wherein the plurality of zones includes a blood zone and further comprises the step of contacting the buffered vaginal fluid with the blood zone to determine whether blood is present in the buffered vaginal fluid.

19. The method for a multi symptom test apparatus according to claim 14, wherein the plurality of zones includes a protein zone and further comprises the step of contacting the buffered vaginal fluid with the protein zone to determine whether protein is present in excess of normal values in the buffered vaginal fluid.

20. The method for a multi symptom test apparatus according to claim 14, wherein the plurality of zones includes a leukocyte zone and further comprises the step of contacting the buffered vaginal fluid with the leukocyte zone to determine whether leukocyte is present in the buffered vaginal fluid.
